(19) United States
(12) Patent Application Publication
(10) Pub. No.: US 2018/0271256 A1
(43) Pub. Date: Sep. 27, 2018

(54) LIP STAMP

(71) Applicant: Toly Management Ltd., Zejtun (MT)

(72) Inventors: Philippe Parker, ATTARD (MT); Nadia Maniscalco, Gudja (MT); Paul Adams, Tarxien (MT); Eralda Gjyrgji, Paola (MT); Brian McNamara, Mosta (MT)

(21) Appl. No.: 15/920,041

(22) Filed: Mar. 13, 2018

Related U.S. Application Data
(60) Provisional application No. 62/477,213, filed on Mar. 27, 2017.

(51) Int. Cl. A45D 40/30; A45D 40/26

(52) U.S. Cl. CPC A45D 40/30; A61K 8/0229; A45D 40/26

(57) ABSTRACT

A lip stamp for applying a cosmetic lip product to a user's lips, including a handle, a rigid base secured to the handle, and an applicator head secured to the base. The periphery of the head has a shape and dimensions corresponding to one-half of a closed human mouth and is bilaterally symmetrical about a straight line corresponding in position to the interface between the lips of the closed mouth. The applicator surface may extend over the entire area within the periphery, or only over an area of an endless strip running along and outwardly bounded by the periphery. A lip cosmetic dispenser includes the lip stamp, a container for a cosmetic lip product into which the applicator head can be inserted to pick up the product for application, and a container lid having an opening releasably holding the lip stamp with the applicator head protected from exposure.

### CROSS REFERENCE TO RELATED APPLICATION

This application claims the benefit, under 35 U.S.C. § 119(e), of U.S. provisional patent application No. 62/477,213 filed Mar. 27, 2017, the entire disclosure of which is incorporated herein by this reference.

### BACKGROUND OF THE INVENTION

This invention relates to applicators for cosmetic lip products, and more particularly to a lip stamp for applying cosmetic products to a human user's lips, as well as to a dispenser including such a lip stamp in combination with a container for the product to be applied.

Cosmetic products as exemplified by lipstick, lip rouge, lip gloss and the like are well known and widely used for application to a human user's lips to enhance or modify lip color and/or shape. Lipstick, for example, is conventionally supplied as a solid or semisolid cylindrical stick that can be exposed endwise from a holder. The user, grasping the opened holder, brings the end of the cosmetic stick into contact with the lips and rubs it over the lips so as to transfer cosmetic material directly from the stick end to the lip surfaces. Thereby the lips are colored (typically reddened) and sharpened in definition from surrounding lipstick-free areas of the face. The natural configuration of the lips can be subtly altered, if desired, by minor deliberate departure of the applied lipstick edge from the natural edge of the lips.

This facile and familiar lipstick-applying procedure, however, is attended with certain drawbacks. In particular, it is difficult to apply the cosmetic directly from the end of a stick thereof with sufficient precision to achieve and accurately position and configure a sharp defining edge of the lipstick-coated area; failure to do so can cause unattractive smearing or irregularity of appearance. These problems are exacerbated by the variation in shape (taper or slope angle and orientation) of the exposed lipstick end that occurs during use as cosmetic material is progressively transferred to the lips and thus removed asymmetrically from the stick end; hence the same manipulative movements of the same stick produce different results depending on how the stick end has been inherently remolded by application of material therefrom.

As an alternative to direct application of lip cosmetic to the lips from the exposed end of a supply stick of the cosmetic itself, it has heretofore been proposed to provide a device with an applicator surface having the general shape of a complete human mouth (upper and lower lips), commonly three-dimensional to conform to lip curvature, and to employ the device for indirect application of cosmetic from a supply thereof to the lips. In use, the cosmetic is first received or picked up by the mouth-form surface of the device, and that surface is then pressed or stamped against the user's lips to transfer the cosmetic from the applicator surface to the lips. Examples of such lip stamp devices are shown in U.S. Pat. Nos. 2,554,965, 7,334,586 and 9,332,819, as well as published international patent application No. WO 2008/013608; related devices also employing a pre-formed applicator of full-lip shape are described in U.S. Pat. No. 2,784,721 and U.S. published patent application No. 2012/0055498.

A significant difficulty presented by these devices arises from the variability in shape and size of lips of different individuals. Any given specific lip-shaped applicator surface is unlikely to conform closely to the lips of a particular user; its use would therefore result in undesirably inaccurate application of lip cosmetic to that user's lip area. Consequently, the described lip stamp and related devices commonly require resort to various expedients for bringing their shapes into proper conformity with an individual user's lips, such as molding, cutting or trimming the applicator to fit the individual, or providing a selection of different lip shapes and sizes from which the user can find the closest match.

### SUMMARY OF THE INVENTION

An object of the present invention is to provide a new and improved lip cosmetic applicator affording facile achievement of superior precision of cosmetic application and sharpness of edge definition of the cosmetic-applied area.

Another object is to provide such an applicator, in particular a lip stamp, which affords these and other advantages with a device configuration broadly and effectively usable by individuals with substantially varying lip shapes and sizes, not requiring individual tailoring of applicator shape to specific users.

A further object is to provide such an applicator characterized by manipulative ease and convenience.

Yet another object is to provide an improved lip cosmetic dispenser including, as applicator, a lip stamp affording the above advantages.

To these and other ends, the present invention, in a first aspect, broadly contemplates the provision of a lip stamp for applying a cosmetic lip product to a user's lips, comprising a rigid handle graspable by a user's hand; a rigid applicator base connected to the handle; and an applicator head, secured to and supported by the base, for conveying and applying cosmetic lip product to a user's lips, wherein the head has a periphery corresponding in configuration and dimensions to a closed human half-mouth constituted of juxtaposed halves of an upper lip and a lower lip. The term “corresponding in configuration and dimensions to” herein means “generally similar to” but not “identical to.”

As a further feature of the invention in this aspect, the periphery of the applicator head is bilaterally symmetrical about a straight line (axis of symmetry) corresponding positionally to an interface between the upper and lower lips of the aforesaid closed human half-mouth. The upper and lower lips of an actual human half-mouth are typically not bilaterally symmetrical about such a line but differ from each other both in peripheral shape and in width (the maximum lip dimension measured transversely of the aforesaid line); hence, while the applicator head of the present invention having this feature corresponds in configuration and dimensions to a human half-mouth, as that term is defined above, it is not identical thereto.

In addition, the periphery of the applicator head advantageously extends along the aforesaid axis of symmetry from a first point to a second point thereon, and has arcuate convex upper and lower outer edges diverging progressively from the aforesaid first point over a major extent (major portion of the length) of the head along the axis of symmetry and then converging to the aforesaid second point. It will be appreciated that these upper and lower edges of the periphery are mirror images of each other, since the applicator head periphery is bilaterally symmetrical about the aforesaid axis.

The applicator head conveniently or preferably has an exposed applicator surface (for bearing cosmetic lip product into contact with the lips) lying in a plane, in which the periphery of the applicator head also lies. The base may have opposed major sides or faces and a base periphery corresponding in configuration to the periphery of the head; the applicator head is secured to one of these faces and the handle projects from the other of the base faces in a direction transverse to (for instance, perpendicular to) the applicator surface plane. The applicator head periphery may be offset inwardly from the base periphery.

In particular embodiments, the applicator surface extends over the entire area bounded by the periphery of the head. In other embodiments, particularly effective as lip lining applicators (i.e., to outline the shape of the lip) but also usable to fill in color, the applicator surface extends only over an area of an endless strip or band running along and outwardly bounded by the periphery; conveniently, in these embodiments, the entire applicator head is an endless strip or band running along and outwardly bounded by the periphery.

The applicator head may be constituted of any material suitable for receiving or picking up cosmetic lip product for application to the user's lips, and for transferring the product from the applicator surface to the lips when pressed and moved against the lips by the user. Advantageously the material of the applicator head is resiliently deformable to facilitate such transfer yet sufficiently self-sustaining to provide a clearly defined and precisely locatable edge of application. For example, the applicator head may be made of a foam material which absorbs the cosmetic product to be applied and releases the applied material when pressed against the lips. Alternatively, the applicator head may be made of an elastomer such as rubber.

The handle and base may be molded integrally of plastic to constitute a unitary rigid structure. The handle preferably projects from the base at a location, along the aforesaid axis of symmetry, at which the upper and lower edges of the applicator periphery are maximally divergent from each other. The lip stamp may also include a protective cap removably engageable with the applicator base for enclosing the applicator head.

In another aspect, the invention embraces a dispenser for a cosmetic lip product, including a lip stamp as described above; a container for a cosmetic lip product, having a neck dimensioned for insertion of the applicator head to pick up a quantity of contained cosmetic lip product for transport to a user's lips; and a removable lid for closing the container neck, the lid having an external opening for removably receiving and holding the applicator base with the handle projecting away from the container and with the applicator head protected from exposure but out of contact with cosmetic lip product in the container. The applicator base may snap-fit into this opening and the lid may be threaded on the container neck.

Further features and advantages of the invention will be apparent from the detailed description hereinafter set forth, together with the accompanying drawings.

### BRIEF DESCRIPTION OF THE DRAWINGS

FIG. 1 is a front external view of a typical or average closed human mouth;

FIG. 2 is a similar view of the applicator head of a lip stamp embodying the present invention in a particular form;

FIGS. 3 and 4 are enlarged perspective views of lip stamps of the present invention respectively having different forms of applicator head;

FIGS. 5, 6 and 7 are views similar to FIG. 2 of the periphery of the applicator head of a lip stamp of the invention, respectively illustrating dimensional and configurational parameters of such periphery for an optimal size of the applicator head (FIG. 5), an exemplary maximum size of such head (FIG. 6) and an exemplary minimum size of such head (FIG. 7), defining an exemplary range of applicator head sizes and shapes for currently preferred embodiments of the invention;

FIG. 8 is an enlarged top plan view of the lip stamp of FIG. 3 illustrating a currently preferred location of the applicator handle relative to the applicator head periphery therein;

FIG. 9 is a perspective view from above of the lip stamp of FIG. 8;

FIGS. 10A, 10B and 10C are, respectively, a perspective view, a side elevational sectional view and a front elevational sectional view of the lip stamp of FIG. 3, having a cover for the applicator head mounted thereon;

FIGS. 11A and 11B are, respectively, a side elevational sectional view and a front elevational sectional view of the lip stamp of FIG. 4, also having a cover for the applicator head mounted thereon;

FIG. 12 is a perspective view of the applicator head of the lip stamp of FIGS. 4, 11A and 11B;

FIG. 13 is an exploded perspective view of the applicator base and cover of the device of FIGS. 10-12;

FIG. 14 is an enlarged perspective view of another lip stamp embodying the invention, in which the applicator head is made of an elastomer (rubber);

FIG. 15 is an exploded perspective view of a dispenser for a cosmetic lip product embodying the invention and including a lip stamp as shown in FIG. 3;

FIG. 16 is a perspective view of another such dispenser embodying the invention; and

FIG. 17 is an enlarged perspective view of a further lip stamp embodying the invention, with an applicator surface for lip lining, in which the applicator head is made of an elastomer (rubber).

### DETAILED DESCRIPTION

The present invention will be described, with reference to the drawings, as embodied in a lip stamp for applying a cosmetic lip product to the lips of a human user, and in a dispenser for such product, including the lip stamp.

A typical or average human mouth 10 to which cosmetic lip products are commonly applied is illustrated in frontal external view in FIG. 1 with the lips closed and essentially expressionless (i.e., not smiling, grimacing, pouting, etc.). References to a closed human mouth herein will be understood to mean a mouth having the attributes, features and mien thus represented in FIG. 1. The closed human mouth 10 consists of an upper lip 11 and a lower lip 12 in contact with each other along a generally horizontal interface line 14, which is not uniformly straight but does not exhibit significant end-to-end curvature. Rectilinear line A-A extending along (and, in FIG. 1, positioned in overlying relation to) this interface line 14, though not conforming exactly thereto throughout its length, is here defined, and will be referred to hereafter, as a straight line corresponding positionally to an interface between the upper and lower lips of a closed human mouth.

As shown, a typical human mouth is not bilaterally symmetrical about line A-A because the upper and lower lips differ from each other in shape and size. The upper lip has an upper edge 16 which curves convexly from the left and right corners 18a, 18b of the mouth toward the center of the mouth but has a small central region 19 of concave curvature forming a “cupid's bow,” while the lower edge 20 of the lower lip has a generally continuous (and more pronounced) convex curvature extending from corner to corner of the mouth (the left corner 18a is of course on the right side of FIG. 1). The width of the lower lip (measured perpendicularly to line A-A, in the plane of FIG. 1) is ordinarily greater than the corresponding dimension of the upper lip. In addition, the two lips have a three-dimensional swell or curvature diverging outwardly from the plane of FIG. 1; and there is some degree of asymmetry between the left and right sides of the typical closed human mouth (on opposite sides of vertical line B-B in FIG. 1).

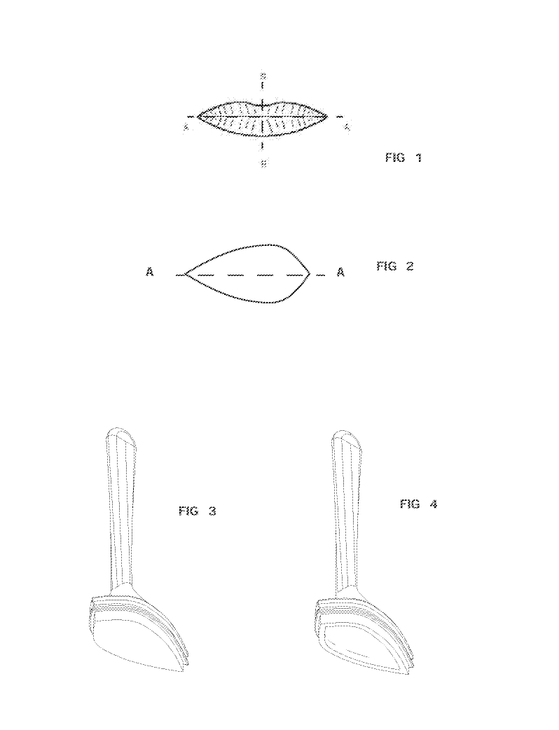

The lip stamp 21 of the invention, in the embodiment of FIG. 3, includes a rigid plastic handle 22 graspable by a user's hand, a rigid plastic applicator base 24 connected (e.g. fixedly) to, or molded integrally with, the handle and an applicator head 28 secured to and supported by the base. Head 28 has an exposed planar applicator surface 30 for conveying and applying a cosmetic lip product to a user's lips.

FIG. 2 illustrates diagrammatically the periphery 32 of the applicator head 28, which is the periphery of the applicator surface 30, in the plane of the applicator surface. This periphery corresponds in configuration and dimensions to a closed human half-mouth constituted of juxtaposed halves of an upper lip 11 and a lower lip 12 (i.e., the half upper lip and half lower lip on the same side C of vertical line B-B in FIG. 1). As FIG. 2 shows, the head periphery 32 is bilaterally symmetrical having, as an axis of symmetry, the aforesaid straight line A-A that corresponds positionally to the interface between the upper and lower lips of a closed human mouth; periphery 32 extends along line A-A from a first point 34 (corresponding to one corner of a closed human mouth) on line A-A to a second point 36 on line A-A, the distance between points 34 and 36 on line A-A being herein termed the length of the periphery 32 and corresponding to the corresponding dimension of a typical human closed half-mouth. More particularly, periphery 32 consists of arcuate convex upper and lower edges (respectively designated 38 and 40) which, in the plane of FIG. 2, diverge progressively from point 34 on line A-A over a major portion (e.g. about 70-75%) of the length of periphery 32 and then converging to line 36 on line A-A. The convex curvature of both edges 38 and 40 is shaped to correspond to the peripheral curvature and width of a lower lip of a human half-mouth, rather than to the less pronounced peripheral curvature and smaller width of a typical upper lip of a human half-mouth. Since the applicator head periphery 32 is bilaterally symmetrical about line A-A, its upper and lower edges 38 and 40 are mirror images of each other; for the same reason, the head periphery 32 is not identical to the periphery of an actual closed human half-mouth.

As hereinafter further explained, the portion of the applicator head 28 extending along line A-A from point 34 to the locality of maximum divergence of edges 38 and 40 corresponds to, and is especially useful for applying cosmetic product to, a user's lips between a corner of the mouth and the central region 19 thereof. The relatively short portion of the head 28 where edges 38 and 40 converge from their locality of maximum divergence to point 36 corresponds to the cupid's bow region of the closed human mouth and facilitates desired application of lip product to that area of the lips.

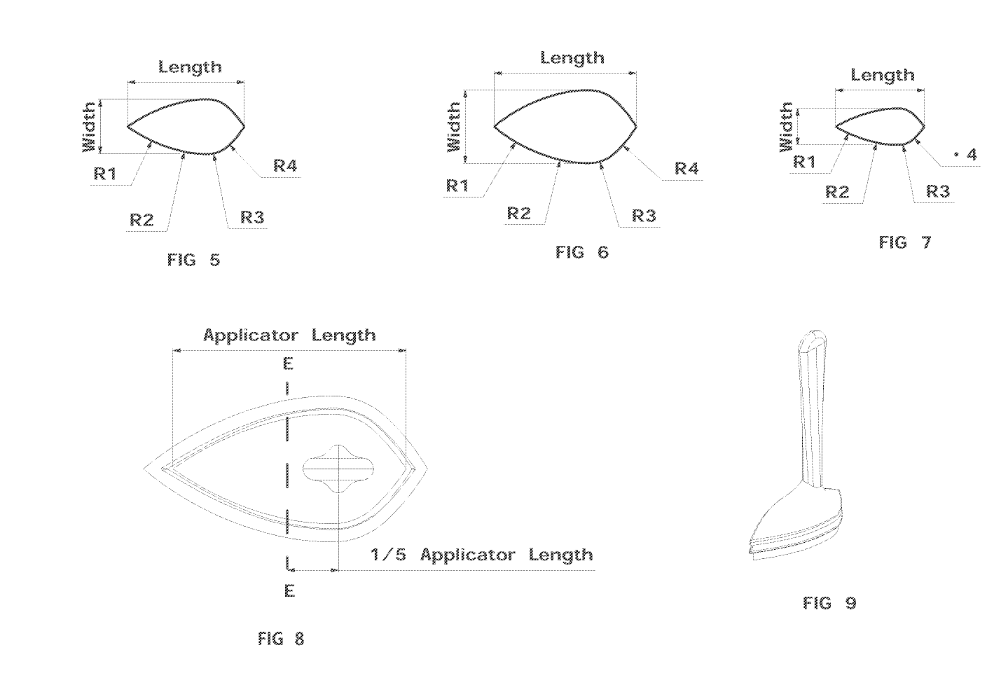

An exemplary range of currently preferred dimensions and configurations of the applicator head periphery 32 is given in FIGS. 5-7, wherein all dimensions are expressed in millimeters. These dimensions, also set forth in TABLE 1 below, include the length of the head periphery 32 along line A-A, the maximum width of the periphery 32 in a direction perpendicular to line A-A in the plane of FIG. 2, and the radius of curvature of the lower edge 40 of the periphery 32 at four locations (respectively designated R1, R2, R3 and R4 in TABLE 1) spaced in succession along edge 40 in the direction from point 34 to point 36, these locations being shown in FIGS. 5-7. FIG. 5 represents currently preferred values (size and shape) for the periphery, FIG. 6 represents exemplary maximum values, and FIG. 7 represents exemplary minimum values. Again owing to the bilateral symmetry of periphery 32 about line A-A, the upper edge 38 of the periphery has the same radii of curvature at the same positions (R1, R2, R3 and R4) along line A-A as the lower edge 40.

TABLE 1
(all dimensions in mm.)

| Dimension | FIG. 7 | FIG. 5 | FIG. 6 |
|---|---|---|---|
| Length | 24.30 | 32.00 | 39.00 |
| Width | 10.00 | 15.00 | 20.00 |
| R1 | 39.04 | 41.54 | 44.04 |
| R2 | 26.69 | 29.19 | 31.69 |
| R3 | 5.35 | 7.85 | 10.35 |
| R4 | 24.60 | 27.10 | 29.60 |

The planar applicator surface 30 is illustrated in FIG. 3 as extending over the entire area bounded by the applicator head periphery 32. In other instances, as in the lip stamp 21′ of FIG. 4, the applicator surface may extend only over an endless strip area along and adjacent to the periphery 32. The head 28′ in FIG. 4 (shown separately in FIG. 12) is an endless strip or band running along and outwardly bounded by the periphery 32′; again, the applicator surface 30′ of this strip lies in a single plane. Exemplary dimensions of this strip or band are a width of 1.5 mm in the plane of the applicator surface 30′ and a thickness of 3.0 mm perpendicular to that plane.

The provision of the applicator head in the form of an endless narrow strip or band, in the embodiment of FIG. 4, is particularly advantageous for use of the lip stamp as a lip lining applicator stamp, i.e., to outline the shape of the lip by a line at the outer edge of the area to which color is applied. It creates an effect beneficial for lip contouring, in that it produces a line that extends downwards creating a lip plumping effect; in addition, the applicator shape point allows the user consistently to produce a good finish at the lip corner, whereas a conventional lip liner has to be continually sharpened in order to achieve the same finish. The lip lining applicator stamp can also be used to fill in color within the defined line, and as thus employed, it enables the user to create an ombre effect (shadow or different colors).

The applicator base 24 of the lip stamp 21 of FIG. 3 (as shown in FIGS. 8-10C and 13) has opposed major sides or faces 42 and 44 and a base periphery 46 corresponding in configuration to the periphery 32 of the head 28 but typically somewhat larger than the head periphery so that the head periphery is offset inwardly from the base periphery as indicated at 48. The head 28 is secured (e.g. adhesively bonded) to the face 44 of the base, with the applicator surface 30 exposed. The handle 22 projects from the other face 42 (which need not be planar but may, for example, slope upwardly to meet the handle), away from the applicator head, in a direction transverse (e.g. perpendicular) to the applicator surface plane. The handle and base may be molded together as an integral rigid structure of any suitable plastic, e.g. acrylonitrile butadiene styrene (ABS). The length and shape of the handle are selected for manipulative convenience and comfort when grasped by the user's hand; an illustrative length is 63.25 mm. from the plane of the applicator surface to the remote extremity of the handle, which may be flat-sided or curved (e.g. lobed) in cross-section.

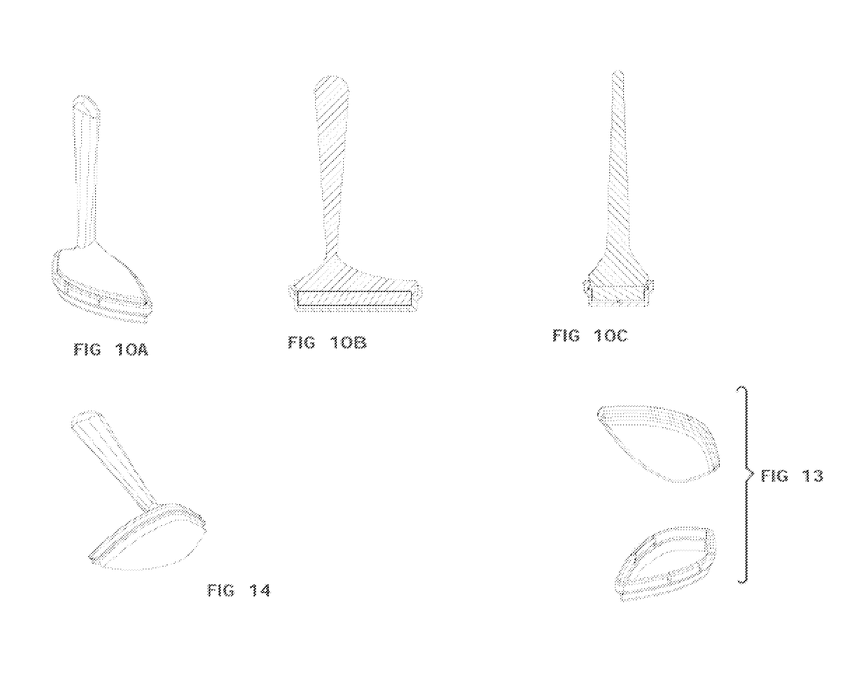

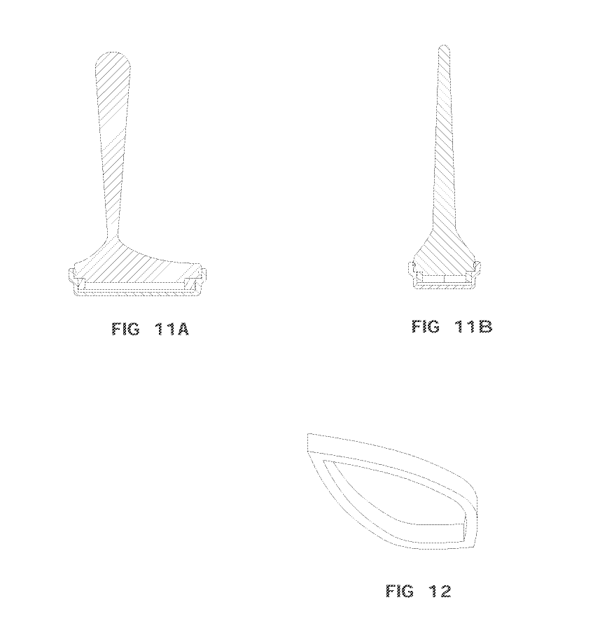

FIGS. 11A and 11B are views, similar to FIGS. 10B and 10C respectively, of the lip stamp 21′ of FIG. 4 in which the applicator head 28′ is an endless strip or band extending along and adjacent to the periphery 32′, having an applicator surface 30′. The face 44 of the base in lip stamp 21 may (as shown in FIGS. 10B and 10C) be formed with a shallow central recess having a periphery conforming to the periphery 32 of the head 28, so that the head seats in this recess while projecting outwardly beyond the face 44 for engaging the lips; the recess contributes to the stability of mounting of the head 28. In the lip stamp 21′ (FIGS. 11A and 11B), a central portion of the face 44′ may protrude above the outer portion of the face 44′, being so shaped as to be snugly surrounded by the head strip or band 28′, again to stabilize the mounting of the head.

Advantageously, to afford the user a clear view (in a mirror, during application of lip cosmetic) of the position of the applicator head relative to the user's lips, the handle 22 is fixed to the base 24 in a position closer to point 36 (corresponding to the center of the lips) than to point 34 (corresponding to a corner of the mouth) along line A-A. A currently preferred handle position is at or near the location (on line A-A) of maximum divergence of the head periphery edges 38 and 40 from each other; this position is shown in FIG. 8 as 70% of the distance from point 34 to point 36 along line A-A, or in other words, with reference to line E-E bisecting the length of the applicator head, a position located at a distance beyond line E-E (in the direction toward point 36 on line A-A) equal to ⅕ of the total length of the periphery 32 along line A-A.

To protect the applicator head 28 against contamination before or between uses, and also to prevent undesired soiling of hands or other surfaces with residual cosmetic product on the applicator surface, a molded plastic applicator cover 50 (FIGS. 10A-13) may be provided. This cover, which need not be completely air-tight, is shaped and dimensioned to enclose the head 28 and has a rim 54 arranged to snap-fit removably over an edge 56 of the base 24 shaped for such engagement with the cover rim.

The material of the applicator head is selected to have a surface suitable to receive or pick up a desired cosmetic lip product to be applied and to convey the product to the user's lips as the user grasps and moves the handle, and to be resiliently deformable so as to apply the product to the lips without smearing (an overly firm surface would smear the product across the skin) yet sufficiently self-sustaining in shape to create a very fine and precise edge line of applied cosmetic. It may be a foam material, such as a synthetic polymeric foam material (one example being NBR, i.e., nitrile rubber, also known as Buna-N, Perbunan, and acrylonitrile butadiene rubber, a synthetic rubber copolymer of acrylonitrile and butadiene). The foam, when used, should be soft enough to pick up and deliver a good dosage of the cosmetic product. The head material may be chosen for compatibility with the specific cosmetic formulation to be applied.

Another applicator head 128, molded as a unitary element of elastomer/rubber, is shown in FIG. 14. This head includes a flange 129 which fits over the edge 56 of the base 24. Again, the periphery of the applicator surface 130 is offset inwardly from the edge of the base and overlying flange 129.

A still further applicator head 128′, formed to serve (like the applicator head 28′ of FIG. 4) as a lip lining applicator and molded (like the head of FIG. 14) as a unitary element of elastomer/rubber, is shown in FIG. 17. This head includes a flange 129′ which fits over the edge 56 of the base 24, with the periphery of the applicator surface 130′ being offset inwardly from the edge of the base and overlying flange 129′. Applicator surface 130′, like that of FIG. 4, extends only over an endless strip area along and adjacent to the head periphery.

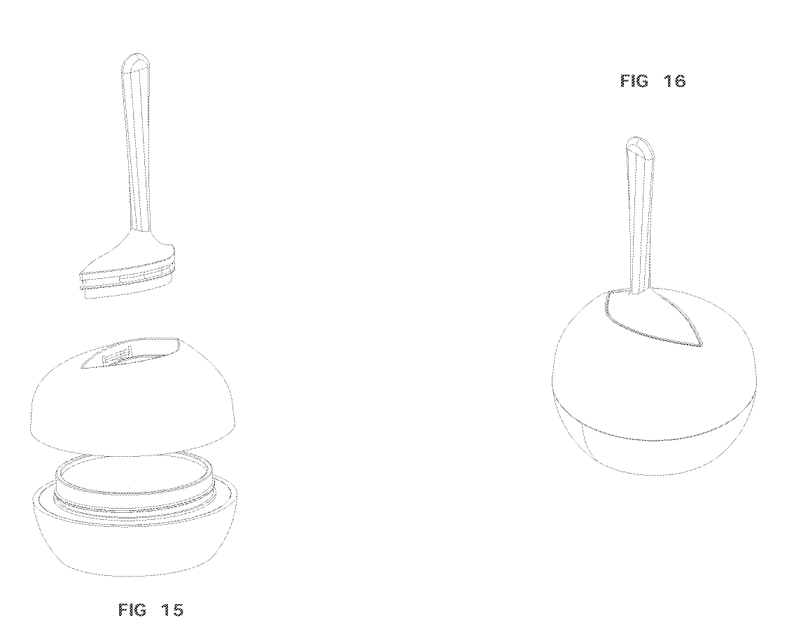

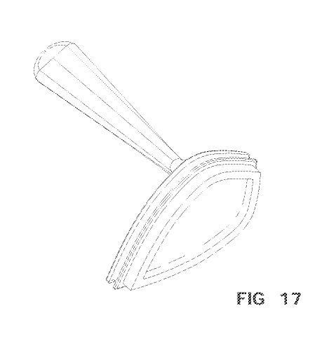

The lip stamp of the invention may be combined with a cosmetic container and lid to constitute a cosmetic lip product dispenser. FIG. 15 illustrates such a dispenser 144, including lip stamp 21, as described above; a cosmetic container 146, having a threaded neck 148 large enough to enable insertion of the lip stamp applicator head 28 therein to pick up cosmetic for application, and a lid 150 that is threaded on the neck to close the container. An opening or socket 152 is formed in the upper outer surface of the lid to removably receive the applicator head and base therein (e.g. by snap-fitting), enclosing and protecting the head between applications while keeping the head out of contact with the cosmetic in the container. Another dispenser 244, structurally and functionally similar to dispenser 144 (i.e., including lip stamp 21, container 246 and lid 250) is shown in FIG. 16.

The use of the lip stamp of the invention, in its above-described embodiments, may now be readily understood. With the applicator head and applicator surface exposed, the user, holding the handle 22 with one hand, causes the lip stamp to pick up a quantity of lip cosmetic on surface 30, for example from a container 146 or other source. The user then manually conveys the lip stamp to the lips, with the surface 30 facing the lips and the point 34 of the surface positioned close to one corner (e.g., right corner 18b, FIG. 1) of the mouth, the surface being oriented so that the axis of symmetry A-A is generally aligned with the long horizontal dimension of the mouth. Thereupon, the user brings the applicator surface into contact with the right half of the lips and manipulates the lip stamp to apply the lip cosmetic on surface 30 to the half-mouth that begins at corner 18b, employing motions including curved motions or gestures which are generally similar to the gestures utilized in conventional lipstick application. The curved edges of the applicator surface periphery enable the user to readily create a precise curved application edge along the lip edges, while the angled peripheral portion adjacent point 36 facilitates desired application to the cupid's bow area and the point 34 allows the user to create a good finish at the lip corner.

More particularly, in applying lip cosmetic with the lip stamp of the invention, the user conveniently operates first on one (say, the upper) right quadrant of the mouth, next on the other (lower) right quadrant of the mouth. Owing to differences in size and shape between the lip stamp applicator surface and a specific user's lips, within each quadrant the user may employ a plurality (2, 3 or more) of stamping motions, selecting and changing the position and/or angle of the lip stamp for each of these stamping motions as dictated by the dimensions and configuration of the lips receiving the cosmetic, to achieve desired completeness and precision of coverage.

Once the first half of the mouth is completed, the user simply inverts the lip stamp (so that point 34 is positioned close to the left mouth corner 18a) and repeats the same application process in succession on the upper left quadrant of the mouth and finally on the lower left quadrant of the mouth. The bilateral symmetry of the applicator surface enables such application with the same device on each side of the mouth. The small (half-mouth) size of the lip stamp avoids problems arising from discrepancies in size and/or shape between the lip stamp and the individual user's lips; although the shape of the lip stamp does not conform exactly to the user's mouth, its arrangement of curvatures enables superior control of precise cosmetic application along the curved lip edges and surfaces. Thus, one size and configuration of applicator is suitable for users generally, despite individual differences in lips. Unlike a conventional lipstick, the lip stamp of the invention maintains its exact same shape throughout use, and over repeated uses.

It is to be understood that the invention is not limited to the features and embodiments hereinabove specifically set forth, but may be carried out in other ways without departure from its spirit.

What is claimed is:

1. A lip stamp for applying a cosmetic lip product to a user's lips, comprising:
a rigid handle graspable by a user's hand;
a rigid applicator base connected to the handle; and
an applicator head secured to and supported by the base, for conveying and applying cosmetic lip product to a user's lips, wherein the head has a periphery corresponding in configuration and dimensions to a closed human half-mouth constituted of juxtaposed halves of an upper lip and a lower lip.

2. A lip stamp as defined in claim 1, wherein the periphery of the head is bilaterally symmetrical having, as an axis of symmetry, a straight line corresponding positionally to an interface between the upper and lower lips of said closed human half-mouth.

3. A lip stamp as defined in claim 2, wherein the periphery of the head extends along said line from a first point on said line to a second point on said line, and has arcuate convex upper and lower outer edges diverging progressively from said first point on said line over a major extent of the head along said line and then converging to said second point on said line.

4. A lip stamp as defined in claim 3, wherein the applicator head has an exposed applicator surface lying in a plane and the periphery of the applicator head also lies in said plane.

5. A lip stamp as defined in claim 4, wherein the base has opposed major faces and a base periphery corresponding in configuration to the periphery of the head, wherein the applicator head is secured to one of said faces and said handle projects from the other of said faces in a direction transverse to said plane.

6. A lip stamp as defined in claim 4, wherein the applicator surface extends over the entire area bounded by the periphery of the head.

7. A lip stamp as defined in claim 4, wherein the handle and the base are molded integrally of plastic.

8. A lip stamp as defined in claim 5, wherein the handle projects from the base in a direction substantially perpendicular to said plane.

9. A lip stamp as defined in claim 5, wherein the handle projects from the base at a location, along said line, at which the upper and lower edges of the applicator periphery are maximally divergent from each other.

10. A lip stamp as defined in claim 4, wherein the periphery of the applicator head is offset inwardly from the base periphery.

11. A lip stamp as defined in claim 1, wherein the applicator head comprises a resiliently deformable material.

12. A lip stamp as defined in claim 1, wherein the applicator head comprises a foam material.

13. A lip stamp as defined in claim 1, wherein the applicator head comprises an elastomer.

14. A lip stamp as defined in claim 1, further including a removable cap engageable with the base for enclosing the applicator head.

15. A lip stamp as defined in claim 1, wherein the applicator surface extends only over an area of an endless strip running along and outwardly bounded by said periphery.

16. A lip stamp as defined in claim 15, wherein the applicator head is an endless strip or band running along and outwardly bounded by said periphery.

17. A lip stamp as defined in claim 15, wherein the periphery of the head is bilaterally symmetrical having, as an axis of symmetry, a straight line corresponding positionally to an interface between the upper and lower lips of said closed human half-mouth; wherein the periphery of the head extends along said line from a first point on said line to a second point on said line, and has arcuate convex upper and lower outer edges diverging progressively from said first point on said line over a major extent of the head along said line and then converging to said second point on said line; and wherein the applicator head has an exposed applicator surface lying in a plane and the periphery of the applicator head also lies in said plane.

18. A lip stamp as defined in claim 15, wherein the base has opposed major faces and a base periphery corresponding in configuration to the periphery of the head, wherein the applicator head is secured to one of said faces and said handle projects from the other of said faces in a direction transverse to said plane; wherein the handle projects from the base in a direction substantially perpendicular to said plane and at a location, along said line, at which the upper and lower edges of the applicator periphery are maximally divergent from each other.

19. A dispenser for a cosmetic lip product, including
a lip stamp as defined in claim 1;
a container for a cosmetic lip product, having a neck dimensioned for insertion of said applicator head to pick up a quantity of contained cosmetic lip product for transport to a user's lips; and
a removable lid for closing said container neck, the lid having an opening for removably receiving the applicator base, with the handle projecting away from the container, to hold the applicator with the head protected from exposure but out of contact with cosmetic lip product in the container.

20. A dispenser as defined in claim 19, wherein the applicator base snap-fits into the base-receiving aperture and the lid is threaded on the container neck.
